(19) United States
(12) Patent Application Publication
(10) Pub. No.: US 2010/0163059 A1
(43) Pub. Date: Jul. 1, 2010

(54) RESTRAINT APPARATUS

(76) Inventors: NEIL TIERNEY, LONDON (GB); RORY WILKINSON, EDINBURGH (GB)

Correspondence Address: DYKAS, SHAVER & NIPPER, LLP, P.O. BOX 877, BOISE, ID, 83701-0877, US

(21) Appl. No.: 11/917,626

(22) PCT Filed: Jun. 15, 2006
(86) PCT No.: PCT/GB06/02187

§ 371 Date: Jun. 5, 2008

(30) Foreign Application Priority Data
Jun. 15, 2005 (GB) 0512111.6

(51) Int. Cl. A61F 5/37

(52) U.S. Cl. 128/876; 128/870

(57) ABSTRACT

A patient restraint apparatus (5) is described. The patient restraint apparatus (5) includes a base (10), at least one restraining means (16) for applying a restraining force to restrain a patient to the base (10), and a plurality of support elements. In use, substantially all the restraining force applied by the restraining means (16) in a downward direction acts on the support elements (20,22) rather than the patient. In one embodiment, the patient restraint apparatus (5) is for use in baby transport incubator.

### FIELD OF THE INVENTION

The present invention relates to a restraint apparatus, restraining a patient in selected positions. The invention relates, particularly but not exclusively, to a restraint apparatus used when a baby is being transported in a transport incubator.

### BACKGROUND OF THE INVENTION

The invention is designed for use with neonates and infants. Throughout the description, the term ‘baby’ shall be used to define both.

The main clinical reasons for the transportation of babies are the complications of prematurity. This stems from the fact that most babies are born in hospitals where there is only general provision for the care of neonates. Those who require intensive care need to be moved to more highly staffed and instrumented intensive care units. While the greatest demand for transport comes from premature babies there are also a significant number of mature babies who may have surgical or related problems and require transportation.

A transport incubator is a system produced or arranged to serve as a complete autonomous unit of care for a baby in a vehicle or craft. The system is typically comprised of a baby compartment which is mounted on a wheeled trolley together with auxiliary clinical devices often comprising infusion pumps and cardiac and respiratory monitoring devices. A baby compartment is commonly defined as an environmentally controlled enclosure intended to contain a baby and with transparent sections which allows for viewing of the baby.

The invention is designed, particularly but not exclusively, for use within the baby compartment of a transport incubator system.

Most transportation is done by land in ambulances. However, helicopters and other forms of air transportation are used when conditions and geography dictate.

Transport incubators are often moved outside hospitals in ambulances. The ambulances used are often old with poor suspension and are very bulky which makes cornering difficult. None of these factors makes for smooth transportation for the baby.

In the worst case scenario ambulances can be involved in a crash scenario. Typically when an ambulance crashes medical staff who travel with the transport incubator are unhurt as they are restrained by their seatbelts. Unfortunately there is no equivalent for the baby within the baby compartment, causing the baby to become a projectile causing serious injury.

The baby is often exposed to excessive vibration during transport from uneven road surfaces being transferred through the vehicle and also from engine vibration. Vibration can have serious physiological effects on the baby. When a baby is inadequately restrained this vibration is further exacerbated and amplified. If it is not restrained the vibration can cause the baby to move independently from the mattress.

The effects of acceleration and deceleration on a baby during transport are exacerbated by inadequate restraint apparatus. Acceleration has serious physiological effects on a baby's cardiovascular system. Blood can pool in dependent areas such as lower limbs. Venous return is reduced. This reduction in preload volume (under filling of the heart) leads to reduced CVP (central venous pressure) and cardiac output. The subsequent fall in BP (blood pressure) can lead to reduced blood flow and perfusion of the baby's vital organs. The normal physiological coping mechanism initiated by a change in vasomotor tone can be lost or impaired by both illness and immaturity. This means that the effects of reduced preload, caused by acceleration, are very likely to affect a sick or premature baby.

The effects of deceleration can have equally serious physiological effects on a baby's cardiovascular system. Blood can pool in the head and upper body. Venous return is increased. This leads to increased cardiac output and CVP. This can cause a rapid increase in BP. Such increases in heart rate may not be a problem in a person with a healthy heart. The sick baby or a baby with a dysfunctional heart may not be able to push their heart rate any further. Such transient stimulus can lead to momentary heart failure.

Transport movement has a seriously detrimental effect on a baby's respiratory system. When a baby is not restrained properly within the baby compartment the twisting and turning movement during transport can cause lung alveoli to become compressed or stretched. This results in ventilation and perfusion mismatching. The incidence is likely to be increased in babies with immature, surfactant deficient lungs. In the short term this will affect ventilatory requirements, gas exchange and oxygenation. In the long term, ventilation and perfusion mismatching has been suggested to increase incidence of chronic lung disease.

Transport also increases the psychological stress on a baby. The baby may experience tachycardia, hypertension and an increase in metabolic rate.

Babies are just as likely to suffer from motion sickness as adults are during transport. The baby may experience nausea, vomiting and feelings of lethargy. Motion sickness is exacerbated by inadequate restraint of the movement of the baby during a transport.

The movement the baby is subjected to during a transport can also affect the auxiliary equipment attached to it. Cables and tubes can become dislodged or even displaced. This can have obvious detrimental effects to the baby's well being. It is an object of the invention to restrain the baby so this cannot happen.

As babies are not restrained adequately at the moment and the resulting detrimental effects this has on the baby during transport the only option available to staff is to get the ambulance to drive slower. This has the obvious effect of a potentially life threatening delay to getting the baby to the appropriate hospital and the level of care and equipment it requires.

In conclusion, the solution to make transport better for the baby is to have it properly restrained.

It is a technical constraint on any solution to this problem for a restraint apparatus to exert almost no pressure onto the baby's body as this can have serious physiological effects. Furthermore minimal contact area and deformation of the baby's natural form is preferable. Babies, particularly premature neonates, have very thin skin which can be damaged easily through the slightest touch and pressure. The restraint apparatus must also allow easy visibility of the baby, easy access to the baby and not interfere with cabling and tubes from auxiliary equipment.

Further technical constraints imposed on any solution to this problem include the desirability for a system to restrain various sizes of babies, in different lying positions for example supine, prone and sideways and to restrain movement of the baby during an ambulance crash situation.

It is important that a device is provided to limit a baby's movement to a defined area within the baby compartment. It has been documented that current systems are inadequate for restraining neonates during transportation.

The restraint devices that are currently available inadequately restrain babies during transport in a transport incubator. Some also cause problems to be exacerbated.

Typically, basic strapping is provided from fixation points on one side of the mattress, over the top of the baby, down to fixation points on the mattress on the other side of the baby. The baby is restrained to the mattress by exerting downwards pressure onto the baby. This can cause physiological damage. Further, these solutions are ineffective as they do not adequately limit the baby's movement either laterally or transversely. They also do not restrain the baby's movement during a crash, resulting in the baby becoming a projectile. They are typically designed only to secure the baby in one position, generally prone, with immovable fixation positions.

Furthermore, existing systems restrict access to the baby by staff and interfere with tubes and cabling from auxiliary equipment. For example, using a net or full body cover restricts the visibility of the baby. The baby needs to be constantly monitored by staff throughout a transfer and the most important method is through observation, particularly of the skin.

Three or five point harnesses currently used do not address immobilisation of the head and their contact area is tight fitting and considerably large causing damage to the babies skin. Restraint of the head is desirable.

It is an object of one embodiment of the present invention to provide a device to help facilitate the transportation of a baby, especially a pre-term neonate, that avoids at least some of the afore-mentioned disadvantages, namely to provide adequate restraint during vibration; acceleration; deceleration; twisting/turning movement.

### SUMMARY OF THE INVENTION

According to a first aspect of the present invention there is provided a patient restraint apparatus comprising:

a base,

at least one restraining means for applying a restraining force to restrain a patient to the base, and

a plurality of support elements,

wherein, in use, substantially all the restraining force applied by the restraining means in a downward direction acts on the support elements rather than the patient.

A patient restraint apparatus, according to an embodiment of the present invention, can be used to restrain a patient, particularly a baby, without applying a downward force directly to the patient. This has the advantage that the patient can be restrained without suffering the injuries and physiological damage which can be caused by downward pressure.

Most preferably, all the restraining force applied by the restraining means acts on the support elements.

Preferably, the support elements are arranged, in use, adjacent the patient.

Preferably, the support elements are arranged to encircle the patient.

Preferably, the support elements are arranged to resist movement of the patient with respect to the base.

Preferably, the support elements are deformable. By using supports elements which can naturally form around a baby's shape it will avoid deformation of the baby's natural form, which can cause discomfort and physiological damage.

Preferably, the support elements are adapted, in use, to abut the patient to resist movement of the patient.

Preferably, the/each restraining means comprises at least one strap.

Preferably, the/each restraining means is adjustable. Adjustable straps permit increased visibility of the patient, easy access to the patient and minimum interference with cabling from auxiliary equipment.

Preferably, the/each restraining means is releasably securable to the base.

Preferably, the base comprises a mattress.

Most preferably, the base comprises a base plate and a mattress.

Preferably, the/each restraining means is releasably securable to the base plate.

Preferably, the base plate is generally rectangular.

The base plate may be part of a transport incubator.

Preferably, the restraint apparatus is adapted to restrain the mattress onto the base plate.

Preferably, the base includes attachment members, the restraining means adapted to be attached to the attachment members.

Preferably, the attachment members are mounted to opposite edges of the base.

Preferably, the attachment members are fitted to opposite sides of the base plate.

Preferably, the attachment members are fixation bars.

Preferably, the fixation bars run longitudinally the length of the base plate.

Preferably, the support elements are pads.

Preferably, the support elements are moveably positioned on an upper surface of the mattress. The support elements may be formed and arranged to provide restraint to a variety of baby sizes and positions.

Preferably, each support element includes an eyelet.

Preferably, the eyelet is provided on the top of the support element.

Preferably, the/each restraining means passes through the eyelet of an associated support element.

Preferably, the support elements are arranged in pairs.

Preferably, in use, the support elements are arranged in pairs on either side of the patient. Such an arrangement provides equal restraint on each side of the patient. Such an arrangement also prevents the patient from twisting and turning from movement during transport.

Preferably, the longitudinal position of the support elements is adjustable by moving the restraining means with respect to the attachment members.

Preferably, the lateral position is adjustable by moving the support elements with respect to the restraining means.

In one embodiment, the longitudinal position of a pad can be adjusted by moving the straps along the fixation bars and the lateral position of a pad can be adjusted either by repositioning the pad or by sliding the pad along the strap.

Preferably, each strap comprises two releasably connected strap sections.

Preferably, each strap section comprises attachment device for attaching to the other strap section.

The attachment device may be a buckle or a hook and loop fastener or the like.

The strap sections may connect over the baby. Alternatively, the strap sections may connect over a support element.

Alternatively, each strap comprises a single strap section.

Preferably, the plurality of support elements include at least one head support element.

Preferably, the at least one head support element includes a first head support element and a second head support element.

Preferably, the at least one head support element includes a head engaging surface.

Preferably, each head engaging surface includes a tapered portion.

Preferably, in use, the first and second head support elements are arranged such that the tapered portions of the first and second head support elements taper towards each other. Having the head engaging surfaces tapering towards each other provides, in use, support for the lower part of the head, the neck and the shoulders, that is, in use with a baby lying on its back, the tapered portions slot in to the sides of the baby's neck to capture the head, engage the neck and be positioned against the shoulders.

Preferably, the plurality of support elements include at least one body support element.

Preferably, the at least one body support element includes a first body support element and a second body support element.

Preferably, the at least one body support element includes a body receiving surface.

Preferably, where there is first and second body support elements, each body support element defines a tapered body receiving surface. When used with a baby in a transport incubator, the head and body support elements prevent the baby from moving during acceleration and deceleration and also resist movement of the baby during a crash.

In one embodiment, the at least one body support element is a feet support element.

Preferably, at least some of the support elements are wedge shaped.

Most preferably, at least some of the support elements are triangular wedge shaped.

Preferably, the wedge shaped support elements define a protruding tip adapted, in use, to extend over the patient for an optimal fit.

Preferably, each support element comprises a first portion and a second portion.

Preferably, the first and second portions comprise materials of differing densities.

Preferably, the first portion is an upper section and the second portion is a lower section.

Preferably, the upper section is a less dense material than the lower section. A less dense material can be more easily compressed then a more dense material.

Preferably, the denser lower section is adapted, in use, to extend in height from the base up to the chest height of the smallest anticipated user. For a transport incubator, this height would be the chest depth of the smallest anticipated baby. Adjustability between the minimum and maximum height of each support element is then created through the compressibility of the upper section. This height adjustability also ensures that a patient, particularly a baby can be transported either on its side or in the prone or supine position. In one embodiment, as the straps are tightened the upper section compresses, creating a decrease in height between the mattress and the strap. The strap is then tightened until it is just touching the baby. This ensures that the baby is restrained yet no pressure is exerted onto the baby from the straps. The pressure from the strap is absorbed by the support element.

Preferably, the support elements comprise a foam or foams.

Alternatively, each support element comprises a sandwich of layers of varying densities. This is an alternative method of creating the necessary adjustability.

In a further alternative, the support element comprises a material of variable density.

Alternatively, or additionally, the support elements include height adjustment means.

Preferably, the height adjustment means are mechanically operated.

Preferably, the height adjustment means includes a ratchet.

Alternatively, the height adjustments means includes one or more of linear bearing, rods or slides or the like.

Preferably, the restraining means restrains the patient sufficiently to ensure the patient stays in constant contact with the base. This feature is particularly useful where the patient may be exposed to vibrations. In one embodiment, straps provide the restraint required in order to ensure a baby stays in constant contact with the mattress during any vibration experienced during transport. This ensures that the baby stays in constant contact with the mattress and no amplification of the vibration occurs.

Preferably, the restraint apparatus is adapted to prevent a baby from coming loose from the restraint apparatus and becoming a projectile during a crash.

Preferably, the restraint apparatus further includes a cover.

Preferably, the cover comprises a base cover and/or support element covers.

Preferably, the cover(s) is removable.

Preferably, the cover(s) is washable.

Preferably, the cover(s) includes a washing bag.

Preferably, the washing bag is attached to the base cover.

Preferably, the washing bag is adapted to receive the base cover and at least one support element cover. A washing bag ensues the covers are kept together during washing.

Preferably, the cover(s) comprises cotton.

Preferably, the support element covers comprise a patient contact surface. For a baby, particularly for the parts of the support elements which contact the baby's skin, the contact surface may comprise a soft material, for example brushed cotton.

Preferably, the mattress comprises a first region and a second region.

Preferably, the first region is adapted to support the patient's head and the second region is adapted to support the patient's body.

Preferably, the first region is softer than the second region.

According to a second aspect of the present invention there is provided a patient restraint apparatus suitable for use in a transport incubator for babies, said restraint apparatus having a patient restraint surface with a head end and a foot end with a longitudinal axis there between, restraining a patient in a generally horizontal lying position, said patient restraint having multiple supports adjustable for a patient lying on their side or any other lying position necessary and for any size of patient from pre term neonate to an infant.

It will be understood that some of the features of the first aspect may be applicable to the second aspect and have not been included here for brevity.

### BRIEF DESCRIPTION OF THE DRAWINGS

Further preferred features and advantages of the present invention will appear from the following detailed description of one embodiment illustrated with reference to the accompanying drawings in which:

FIG. 1 shows a perspective view of an embodiment of the invention, and

FIG. 2, comprising FIGS. 2a and 2b, shows perspective views of the adjustable body and head support elements.

### DETAILED DESCRIPTION OF THE DRAWINGS

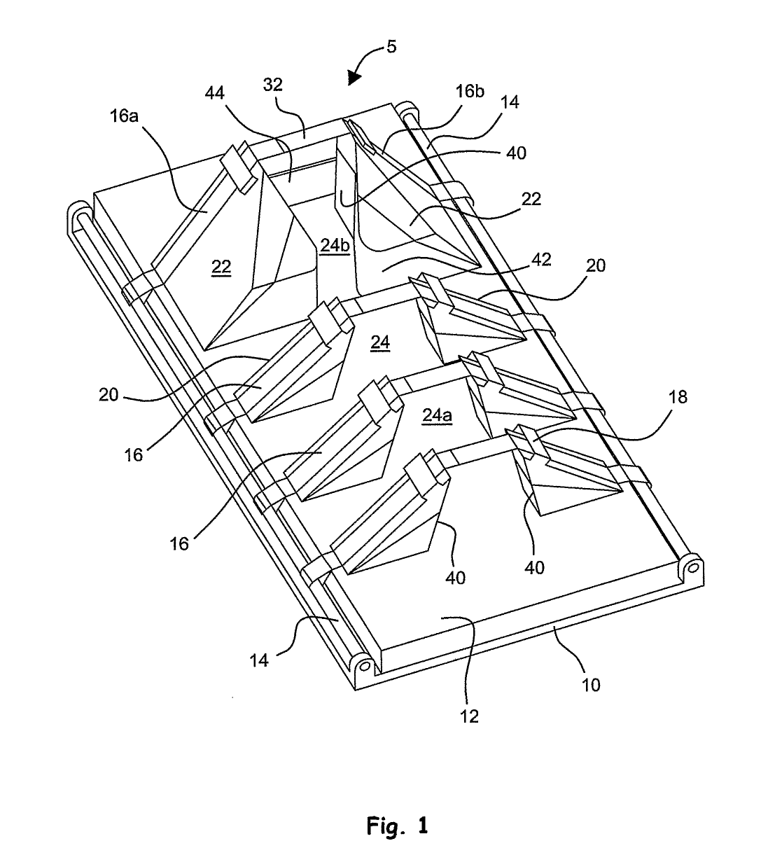

FIG. 1 shows a perspective view of a restraint apparatus, generally indicated by reference numeral 5, according to an embodiment of the present invention for use in a transport incubator. The restraint apparatus 5 has a base plate 10 upon which a mattress 12 rests. The restraint apparatus 5 further includes six adjustable body support elements 20, two adjustable head support elements 22 and restraining means in the form of four straps 16. The straps 16 are attached to first and second fixation bars 14 mounted on the sides of the base plate 10. Each strap 16 is associated with a pair of support elements 20,22.

As can be seen from FIG. 1, the support elements 20,22 are arranged as three pairs of body support elements 20 and a pair of head support elements 22. The support elements 20,22 define a baby receiving region 24 adapted to receive a baby (not shown). The baby receiving region 24 comprises a body receiving region 24a between the body support elements 20 and a head receiving region 24b between the head support elements 22.

In use, a baby lies on the mattress 12 and the support elements 20,22 are arranged such that engagement surfaces 40 defined by the body and head support elements 20, 22 abut and restrain the baby. It will be noted that each engagement surface 40 defined by the head support elements 22 includes a tapered portion 42 which tapers inwardly towards, in use, the baby's neck.

Each strap 16 passes through an eyelet 18 located upon each of the strap's associated pair of support elements 20,22. Each strap 16 is in two parts 16a,16b, the two parts 16a,16b meet in the middle over the baby receiving region 24, and are connected by means of a hook and loop fastener 32. An additional restraint strap 44 is provided across the gap between the head support elements 22. The additional strap 44 is fixed to the end surfaces of each head support element 22 and is a further restraint preventing the baby from moving in the event of a crash during transit of the baby.

To select the desired lateral position of the support elements 20,22 the support elements 20,22 are moved along the straps 16.

To select the desired longitudinal position of the support elements 20,22, the straps 16 are moved along the fixation bars 14.

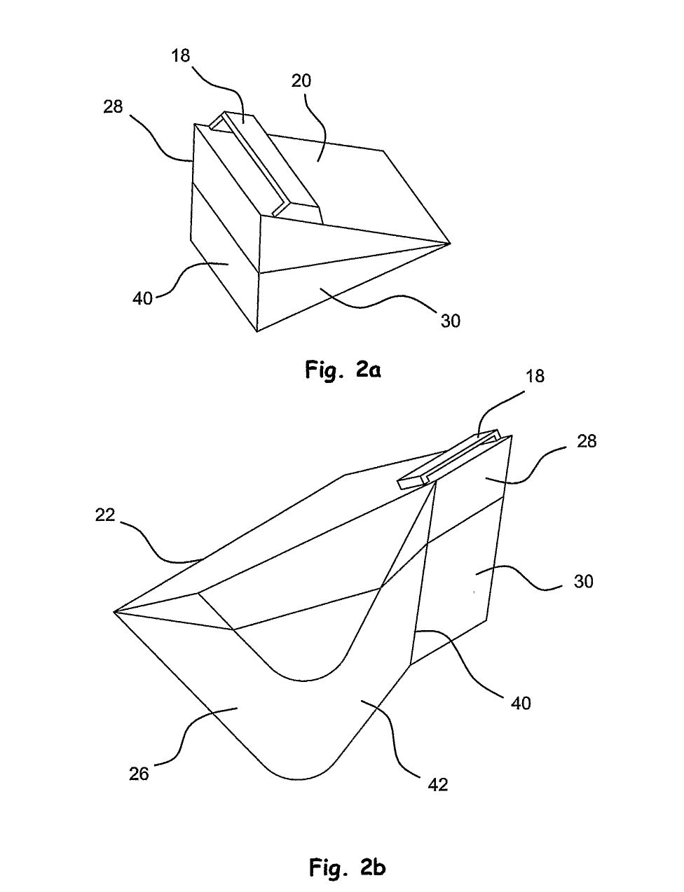

Referring now to FIG. 2, comprising FIGS. 2a and 2b, perspective views of the adjustable body and head support elements 20,22 of FIG. 1, the tapered portion 42 of the head support element engagement surface 40 can be seen more clearly. The engagement surface 40 also includes a shoulder engaging portion 26 which can abut the baby's shoulders (if the baby is lying on its back).

To select the desired height of the straps 16 over the baby's chest, the upper portion 28 of each support element 20,22 is constructed from a low density material which allows adjustability through compressibility. The lower portion 30 of the each support element 20,22 is constructed from a high density material which is still compliant for the baby's comfort. The lower portion 30 performs the function of dictating a minimum strap height which is related to the minimum chest depth of the smallest patients placed in the restraint apparatus 10.

To use the restraint apparatus 5, a baby is lowered onto the mattress 12 and the head and body support elements 20,22 are positioned around the baby. The strap sections 16a,16b are threaded through the eyelets 18 and each strap section 16a,16b is tightened down on its respective support element 20,22, compressing the support element 20,22 until the strap sections 16a,16b are just touching the baby. The strap sections 16a,16b are then connected by the hook and loop fastener 32. This arrangement ensures that the baby is restrained yet no pressure is exerted onto the baby from the straps 16. The pressure from the straps 16 is absorbed by the support elements 20,22.

It will be appreciated that various modifications may be made to the above described embodiments without departing from the scope of the invention. For example the construction of the support elements could be of a mono material with variable density or the adjustment in height of the support elements could be by mechanical means.

1. A patient restraint apparatus comprising:
a base,
at least one restraining means for applying a restraining force to restrain a patient to the base, and
a plurality of support elements,
wherein, in use, substantially all the restraining force applied by the restraining means in a downward direction acts on the support elements rather than the patient.

2. A patient restraint apparatus according to claim 1, wherein the restraining force applied by the restraining means acts on the support elements.

3. A patient restraint apparatus according to claim 1, wherein the support elements are arranged, in use, adjacent the patient.

4. A patient restraint apparatus according to claim 1, wherein at least some of the support elements are arranged to encircle the patient.

5. A patient restraint apparatus according to claim 1, wherein the support elements are arranged to resist movement of the patient with respect to the base.

6. A patient restraint apparatus according to claim 1, wherein the support elements are deformable.

7. A patient restraint apparatus according to claim 1, wherein the support elements are adapted, in use, to abut the patient to resist movement of the patient.

8. A patient restraint apparatus according to claim 1, wherein, in use, the support elements are arranged in pairs on either side of the patient.

9. A patient restraint apparatus according to claim 1, wherein said restraining means comprises at least one strap.

10. A patient restraint apparatus according to claim 9, wherein said strap comprises two releasably connected strap sections.

11. A patient restraint apparatus according to claim 10, wherein each strap section comprises attachment device for attaching to the other strap section.

12. A patient restraint apparatus according to claim 1, wherein said restraining means is adjustable.

13. A patient restraint apparatus according to claim 1, wherein said restraining means is releasably securable to the base.

14. A patient restraint apparatus according to claim 1, wherein the base comprises a mattress.

15. A patient restraint apparatus according to claim 1, wherein the base comprises a base plate and a mattress.

16. A patient restraint apparatus according to claim 14, wherein the support elements are moveably positioned on an upper surface of the mattress.

17. A patient restraint apparatus according to claim 14, wherein the mattress comprises a first region and a second region.

18. A patient restraint apparatus according to claim 17, wherein the first region is adapted to support the patient's head and the second region is adapted to support the patient's body.

19. A patient restraint apparatus according to claim 17, wherein the first region is softer than the second region.

20. A patient restraint apparatus according to claim 15, wherein said restraining means is releasably securable to the base plate.

21. A patient restraint apparatus according to claim 1, wherein the base includes attachment members, the restraining means adapted to be attached to the attachment members.

22. A patient restraint apparatus according to claim 21, wherein the attachment members are mounted to opposite edges of the base.

23. A patient restraint apparatus according to claim 1, wherein said bases further comprises a base plate and a mattress, said base further comprises attachment members, a restring means adapted to attach to said attachment members, and attachment members are fitted to opposite sides of the base plate.

24. A patient restraint apparatus according to claim 21, wherein the attachment members are fixation bars.

25. A patient restraint apparatus according to claim 1, wherein said base further comprise a base plate and a mattress, said base further comprises attachment members, a restricting means adapted to attach to said attachment members, and said attachment members are mounted to opposites edges of said base, the fixation bars run longitudinally the length of the base plate.

26. A patient restraint apparatus according to claim 1, wherein each support element includes an eyelet.

27. A patient restraint apparatus according to claim 26, wherein the eyelet is provided on the top of the support element.

28. A patient restraint apparatus according to claim 26, wherein the/each restraining means passes through the eyelet of an associated support element.

29. A patient restraint apparatus according to claim 21, wherein the longitudinal position of the support elements is adjustable by moving the restraining means with respect to the attachment members.

30. A patient restraint apparatus according to claim 1, wherein the lateral position of the support elements is adjustable by moving the support elements with respect to the restraining means.

31. A patient restraint apparatus according to claim 1, wherein the plurality of support elements include at least one head support element.

32. A patient restraint apparatus according to claim 31, wherein the at least one head support element includes a first head support element and a second head support element.

33. A patient restraint apparatus according to claim 32, wherein each head support element includes a head engaging surface, the head engaging surfaces including a tapered portion.

34. A patient restraint apparatus according to claim 33, wherein, in use, the first and second head support elements are arranged such that the tapered portions of the first and second head support elements taper towards each other.

35. A patient restraint apparatus according to claim 1, wherein the plurality of support elements include at least one body support element.

36. A patient restraint apparatus according to claim 1, wherein at least some of the support elements are wedge shaped.

37. A patient restraint apparatus according to claim 1, wherein at least some of the support elements are triangular wedge shaped.

38. A patient restraint apparatus according to claim 37, wherein the wedge shaped support elements define a protruding tip adapted, in use, to extend over the patient for an optimal fit.

39. A patient restraint apparatus according to claim 1, wherein each support element comprises a first portion and a second portion.

40. A patient restraint apparatus according to claim 39, wherein the first and second portions comprise materials of differing densities.

41. A patient restraint apparatus according to claim 39 wherein the first portion is an upper section and the second portion is a lower section.

42. A patient restraint apparatus according to claim 41, wherein the upper section is a less dense material than the lower section

43. A patient restraint apparatus according to claim 42, wherein the denser lower section is adapted, in use, to extend in height from the base up to the chest height of the smallest anticipated user.

44. A patient restraint apparatus according to claim 1, wherein the support elements comprise a foam or foams.

45. A patient restraint apparatus according to claim 1, wherein each support element comprises a sandwich of layers of varying densities.

46. A patient restraint apparatus according to claim 1, wherein the support element comprises a material of variable density.

47. A patient restraint apparatus according to claim 1, wherein the support elements include height adjustment means.

48. A patient restraint apparatus according to claim 47, wherein the height adjustment means are mechanically operated.

49. A patient restraint apparatus according to claim 1, wherein the restraining means restrains the patient sufficiently to ensure the patient stays in constant contact with the base.

50. A patient restraint apparatus according to claim 1, wherein the restraint apparatus further includes a cover.

51. A patient restraint apparatus according to claim 50, wherein the cover comprises a base cover and/or support element covers.

52. A patient restraint apparatus according to claim 50, wherein the cover is removable.

53. A patient restraint apparatus according to claim 50, wherein the cover is washable.

54. A patient restraint apparatus according to claim 50, wherein the cover(s) includes a washing bag.

55. A patient restraint apparatus according to claim 54, wherein the washing bag is attached to the base cover.

56. A patient restraint apparatus according to claim 54, wherein the washing bag is adapted to receive the base cover and at least one support element cover.

57. A patient restraint apparatus according to claim 50, wherein the cover comprises cotton.

58. A patient restraint apparatus according to claim 51, wherein the support element covers comprise a patient contact surface.

59. A patient restraint apparatus according to claim 58, wherein the contact surface comprises a soft material.

60. A patient restraint apparatus suitable for use in a transport incubator for babies, said restraint apparatus having a patient restraint surface with a head end and a foot end with a longitudinal axis there between, restraining a patient in a generally horizontal lying position, said patient restraint having multiple supports adjustable for a patient lying on their side or any other lying position necessary and for any size of patient from pre term neonate to an infant.
